(19) United States
(12) Patent Application Publication
(10) Pub. No.: US 2024/0424262 A1
(43) Pub. Date: Dec. 26, 2024

(54) Anti-ingrowth Cathlock

(71) Applicant: Bard Peripheral Vascular, Inc., Franklin Lakes, NJ (US)

(72) Inventors: Christian Andersen, Queen Creek, AZ (US); Bryon Ray Densley, Rochester, MN (US); Diana Fiumefreddo, Salt Lake City, UT (US)

(21) Appl. No.: 18/690,240

(22) PCT Filed: Sep. 10, 2021
(86) PCT No.: PCT/US21/49888

§ 371 Date: Mar. 7, 2024

(51) Int. Cl. A61M 25/04; A61M 39/02

(52) U.S. Cl. CPC A61M 25/04; A61M 39/0247; A61M 2039/0273; A61M 2039/0276; A61M 2039/0282

(57) ABSTRACT

Embodiments disclosed herein are directed to an anti-ingrowth cathlock including a compliant gasket. The cathlock can engage an outer surface of a catheter and secure the catheter to a stem of a port, or similar vascular access device, The gasket can be compressed between the cathlock and one of the catheter, stem, or port body to form a continuous outer profile therebetween and mitigate tissue ingrowth. Due to the tolerances during manufacture of the catheter, cathlock and port, gaps can occur between these portions when assembled, allowing for tissue ingrowth and complicating explantation of the port/catheter/cathlock assembly. The compliant gasket can be disposed on one of the port or the catheter and can fill these gaps, mitigating tissue ingrowth.

### SUMMARY

Briefly summarized, embodiments disclosed herein are directed to an anti-ingrowth cathlock device and associated methods thereof. The anti-ingrowth cathlock device can include a compliant, soft polymer configured to elastically deform when compressed between the resilient cathlock and one or more of the catheter, stem, port body or portions thereof. The compliant polymer portion in the deformed state expands to fill any potential gaps between the cathlock and one or more of the catheter, stem, port body or portions thereof, providing a continuous outer profile therebetween and mitigating tissue ingrowth. Advantageously, the anti-ingrowth cathlock device can facilitate explantation of the catheter and port assembly.

Disclosed herein is a cathlock system including, a port including a stem extending therefrom, a catheter defining a lumen and configured to engage the stem, a cathlock configured to encircle a portion of one or both of the stem and the catheter, and a gasket formed of a compliant material and configured to elastically transition between an undeformed state and a deformed state, the gasket in the deformed state forming a continuous outer profile between an outer surface of the cathlock and one or both of the catheter and the port.

In some embodiments, the gasket is coupled with the port body and extends annularly about the stem. In some embodiments, the gasket defines a disc shape, extending along a plane perpendicular to a longitudinal axis of the stem, an outer diameter of the gasket in the undeformed state is equal to, or less than, an outer diameter of the cathlock. In some embodiments, the outer diameter of the gasket in the deformed state is equal to or larger than the outer diameter of the cathlock. In some embodiments, the outer diameter of the gasket in the deformed state is larger than the outer diameter of the gasket in the undeformed state.

In some embodiments, the gasket is coupled to an inner surface of the cathlock and extends between a distal end and a proximal end of the cathlock. In some embodiments, the gasket further includes a flange extending radially outward from a proximal end of the gasket. In some embodiments, the flange defines a first diameter in the undeformed state, and a second diameter in the deformed state, the second diameter being larger than the first diameter. In some embodiments, the first diameter of the flange is less than an outer diameter of the cathlock, and the second diameter of the flange is equal to, or larger than the outer diameter of the cathlock.

In some embodiments, the cathlock is formed of a first material and the gasket is formed of a second material different from the first material. In some embodiments, the first material defines a first durometer and the second material defines a second durometer, the second durometer being less than the first durometer. In some embodiments, the first material is a substantially rigid material and the second material is an elastically deformable material. In some embodiments, the first material includes one of a plastic, polymer, metal, alloy, or composite, and the second material includes one of a plastic, polymer, elastomer, thermoplastic elastomer (“TPE”), rubber, or silicone rubber.

In some embodiments, the gasket is formed of a biocompatible, or inert, material and is configured to mitigate tissue ingrowth between the cathlock and one or both of the catheter and the port. In some embodiments, the cathlock system further includes a valve disposed between an outer surface of the catheter and an inner surface of the cathlock and configured to provide a fluid tight seal therebetween.

Also disclosed is a system for coupling a catheter to a subcutaneous vascular access device including, a body of the subcutaneous vascular access device having a recess disposed therein, a stem extending longitudinally from the recess, a catheter defining a lumen and configured to engage the stem, a cathlock configured to encircle a portion of one or both of the stem and the catheter, a proximal end of the cathlock configured to fit within the recess, and a gasket formed of a compliant material and configured to elastically transition between a deformed state and an undeformed state, the gasket in the deformed state forming a continuous outer profile between an outer surface of the cathlock and the catheter.

In some embodiments, the cathlock includes a flared portion disposed at a proximal end of the cathlock. In some embodiments, the flared portion co-operates with an outer surface of the body to define a continuous outer profile when the proximal end of the cathlock is disposed within the recess. In some embodiments, the gasket is coupled to an inner surface of the cathlock and extends between a distal end and the proximal end of the cathlock. In some embodiments, the gasket is formed integrally with the cathlock. In some embodiments, the gasket is formed as a separate structure and coupled with the cathlock with an interlocking fit, interference fit, press-fit, or snap fit engagement, adhesive, bonding, or welding.

In some embodiments, the cathlock is formed of a first material and the gasket is formed of a second material different from the first material. In some embodiments, the first material defines a first durometer and the second material defines a second durometer, the second durometer being less than the first durometer. In some embodiments, the cathlock system further includes a valve disposed between an outer surface of the catheter and an inner surface of the cathlock and configured to provide a fluid tight seal therebetween.

Also disclosed a method of coupling a catheter to a stem of a port including, urging a proximal portion of the catheter over the stem of the port, slidably engaging a cathlock over the proximal portion of the catheter, elastically deforming a gasket to a deformed state to provide a continuous outer profile between an outer surface of the cathlock and one or both of the catheter and the port.

In some embodiments, the gasket is coupled with the port body and defines a first outer diameter in the deformed state and a second outer diameter in an undeformed state, the first outer diameter being greater than the second outer diameter and equal to, or greater than, an outer diameter of the cathlock. In some embodiments, the gasket is coupled to an inner surface of the cathlock and extends between a distal end and a proximal end of the cathlock. In some embodiments, the method further includes compressing a flange, between the cathlock and the port, the flange extending radially outward from a proximal end of the gasket.

In some embodiments, the outer diameter of the flange in the deformed state is equal to, or larger than, the outer diameter of the cathlock. In some embodiments, the cathlock is formed of a first material and the gasket is formed of a second material different from the first material. In some embodiments, the first material defines a first durometer and the second material defines a second durometer, the second durometer being less than the first durometer. In some embodiments, the first material is a substantially rigid material and the second material is an elastically deformable material. In some embodiments, the method further includes a valve disposed between an outer surface of the catheter and an inner surface of the cathlock and configured to provide a fluid tight seal therebetween.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A shows a plan view of a port, catheter, and cathlock assembly of a vascular access system, in accordance with embodiments disclosed herein.

FIG. 1B shows a cross-section view of a port, catheter, and cathlock assembly of a vascular access system, in accordance with embodiments disclosed herein.

FIG. 2A show a plan view of a port and catheter assembly including an anti-ingrowth cathlock, in accordance with embodiments disclosed herein.

FIG. 2B shows a cross-section view of a port and catheter assembly including an anti-ingrowth cathlock, in accordance with embodiments disclosed herein.

FIG. 2C shows an exploded perspective view of a port and catheter assembly including an anti-ingrowth cathlock, in accordance with embodiments disclosed herein.

FIGS. 2D-2F show close up detail of a gasket of an anti-ingrowth cathlock, in accordance with embodiments disclosed herein.

FIGS. 3A-3B show perspective views of an anti-ingrowth cathlock, in accordance with embodiments disclosed herein.

FIGS. 4A-4C show an exemplary method of use for an anti-ingrowth cathlock, in accordance with embodiments disclosed herein.

FIG. 5A shows a perspective view of a port, catheter and anti-ingrowth cathlock, in accordance with embodiments disclosed herein.

FIG. 5B shows a plan view of a port, catheter and anti-ingrowth cathlock, in accordance with embodiments disclosed herein.

FIG. 5C shows a perspective view of an anti-ingrowth cathlock, in accordance with embodiments disclosed herein.

FIG. 5D shows a cross-section view of a port, catheter and anti-ingrowth cathlock, in accordance with embodiments disclosed herein.

FIGS. 6A-6C show an exemplary method of use for an anti-ingrowth cathlock, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

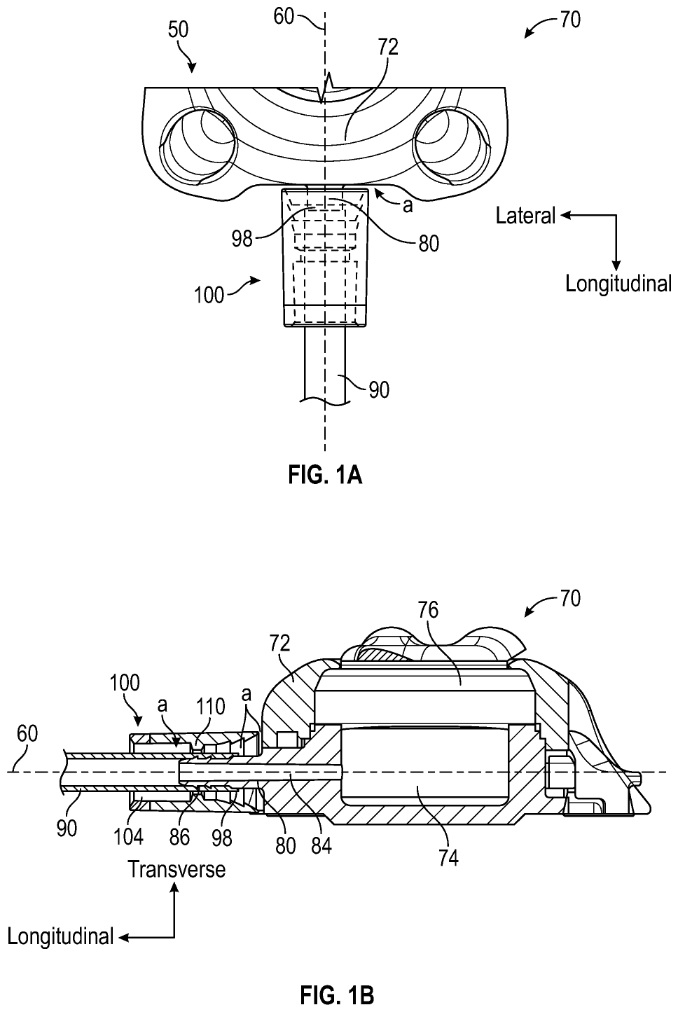

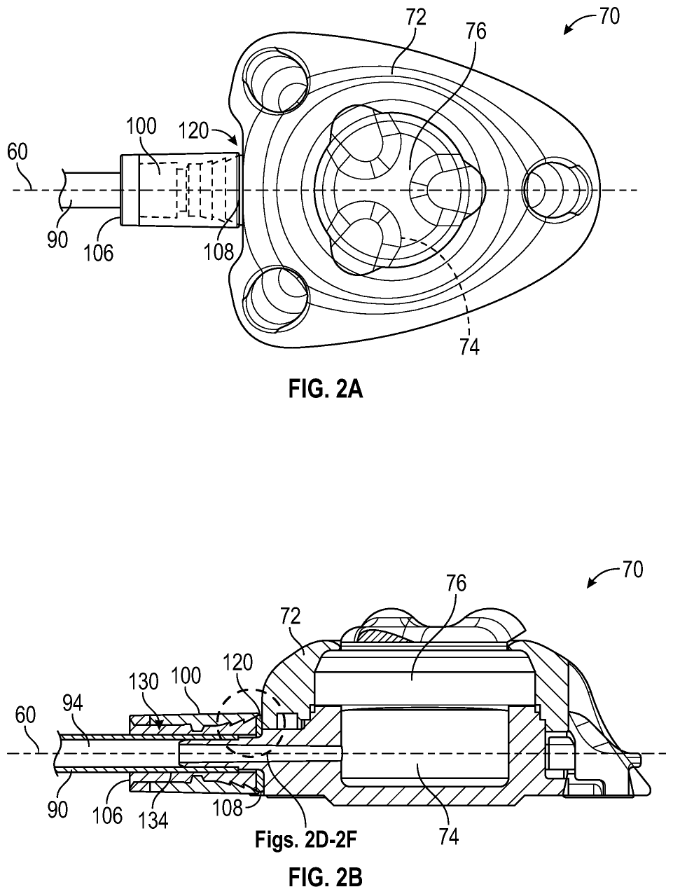

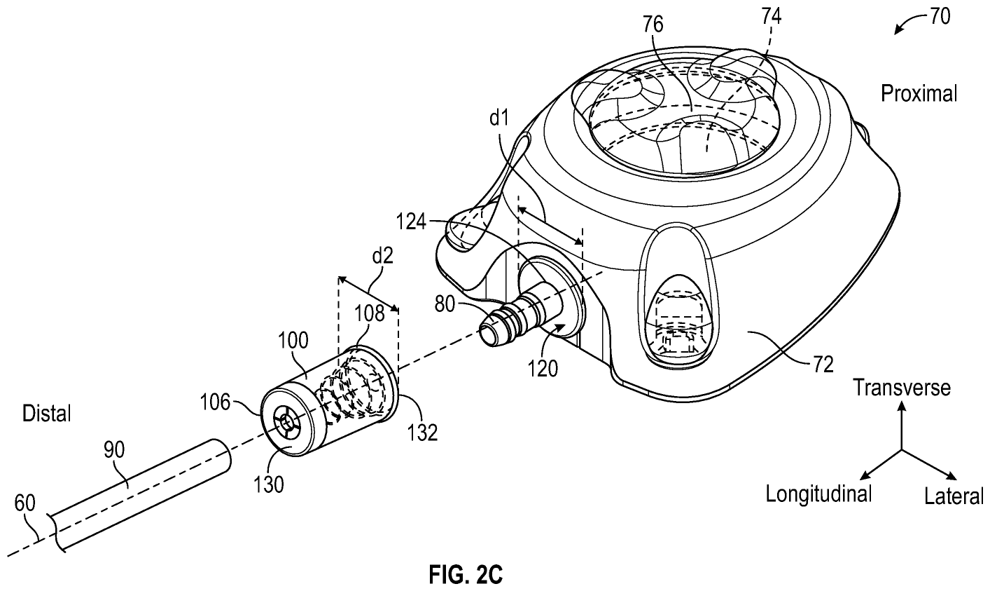

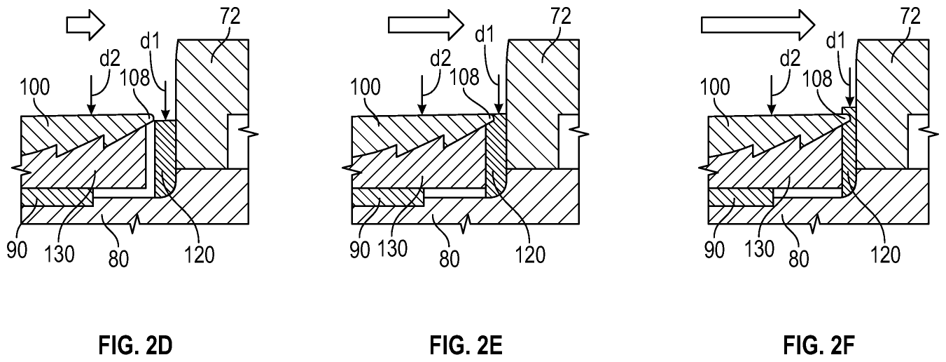

To assist in the description of embodiments described herein, as shown in FIGS. 1A-1B, 2C a longitudinal axis extends substantially parallel to an axial length of the catheter. A lateral axis extends normal to the longitudinal axis, and a transverse axis extends normal to both the longitudinal and lateral axes. As used herein, a horizontal plane extends along the lateral and longitudinal axes. A vertical plane extends normal to the horizontal plane.

Unless defined otherwise, all technical and scientific terms used herein have the same meaning as commonly understood by those of ordinary skill in the art.

FIGS. 1A-1B show an exemplary vascular access system 50 generally including a port 70, catheter 90 and a cathlock device (“cathlock”) 100. The cathlock 100 can be configured to secure the catheter 90 to a stem 80 of the port 70, or similar medical device. More specifically, the cathlock 100 can slidably engage an outer surface of the catheter 90 and can compress a portion of the catheter 90 on to the stem 80 of the port 70 to provide a fluid-tight engagement therebetween.

In an embodiment, the catheter 90 can include a catheter body 92 defining a catheter lumen 94 and extending between a distal tip and a proximal end 98. In an embodiment, the proximal end 98 of the catheter 90 can be coupled to the stem 80 by urging the catheter 90 axially thereover. In an embodiment, the proximal end 98 of the catheter 90 can be trimmable prior to coupling the catheter 90 with the stem 80. In an embodiment, the catheter 90 can be formed of a flexible or compliant material configured to elastically deform radially outwards and engage the stem 80 in an interference fit. In an embodiment, the catheter 90 can be formed of a plastic, polymer, elastomer, thermoplastic elastomer (“TPE”), rubber, silicone rubber, or the like.

In an embodiment, the port 70 can generally include a body 72 defining a reservoir 74 that is in fluid communication with a lumen 84 of the stem 80. The port 70 can further include a needle penetrable septum 76 configured to provide access to the reservoir 74. In an embodiment, an access needle can extend percutaneously, through the needle-penetrable septum 76 and into the reservoir 74 to provide fluid communication therewith. As will be appreciated, the port 70 is an exemplary medical device in fluid communication with the stem 80 and embodiments described herein can be used with various medical devices that include a rigid stem 80 coupled to a compliant catheter 90.

In an embodiment, the cathlock 100 can define a cathlock lumen 104, extending longitudinally, and configured to receive one or both of a portion of the catheter 90 and a portion of the stem 80 therein. The cathlock 100 can slidably engage an outer surface of the catheter 90 and compress a portion of the catheter 90 onto the stem 80. The cathlock 100 can engage the catheter 90/stem 80 assembly in an interference fit, press-fit, or snap-fit engagement. In an embodiment, the cathlock 100 can be formed of a resilient or substantially rigid material, defining a first durometer, and can include a plastic, polymer, metal, alloy, composite, combinations thereof, or the like.

In an embodiment, the cathlock 100 can include a rim 110 or similar structure extending radially inward from an inner surface of the cathlock lumen 104. The rim 110 can be configured to engage a barb 86 or similar structure disposed on the stem 80. With the catheter 90 disposed over the barb 86 of the stem 80, the rim 110 can engage the barb 86 in an interference fit, press-fit, or snap-fit engagement, with a portion of the catheter 90 disposed therebetween.

As shown in FIGS. 1A-1B, due to the tolerances in the manufacturing of one or more of the cathlock 100, catheter 90, or port 70, gaps (a) can occur between one or more of the cathlock 100, catheter 90, or port 70, or combinations thereof. When placed subcutaneously, tissues can grow into these gaps (a) binding and encapsulating the cathlock 100, catheter 90, port 70 assembly. As such, explantation of the cathlock 100, catheter 90, port 70 assembly can be time consuming, requiring additional cutting, forming scar tissue, and increasing recovery times.

In an embodiment, as shown in FIGS. 2A-2C, the anti-ingrowth cathlock 100 can include one or more gaskets, for example a first, port gasket 120 and a second, cathlock gasket 130. The gaskets 120, 130 can be formed of a compliant material, defining a second durometer, and can include a plastic, polymer, elastomer, rubber, silicone rubber, thermoplastic elastomer (“TPE”), combinations thereof, or the like. The second durometer can be less, or softer, than the first durometer. In an embodiment, the gasket 120, 130 can be formed of a biocompatible material. In an embodiment, the gasket 120, 130 can be formed of an inert material.

In an embodiment, the gaskets 120, 130 can extend about a central longitudinal axis 60 and can be configured to provide a continuous outer surface between one or more of the catheter 90, port 70, and the cathlock 100. As used herein, a “continuous outer profile” can include a substantially even surface or convex surface extending between one or more of the catheter 90, port 70, and the cathlock 100. In an embodiment, the gaskets 120, 130 can be formed as a separate structure and coupled with one of the cathlock 100, stem 80, or port body 72 in an interference fit engagement, press-fit, or snap fit engagement, or the like. In an embodiment, one of the cathlock 100, stem 80, or port body 72 can include a recess configured to receive a portion of the gasket 120, 130 therein and retain the gasket 120, 130 in an interlocking fit, interference fit, press-fit, or snap fit engagement, or the like. In an embodiment, the gasket 120, 130 can coupled with one of the cathlock 100, stem 80, or port body 72 with adhesive, bonding, welding, or the like. In an embodiment, the gasket 120, 130 can be formed integrally with the cathlock 100, for example in a two shot injection mold process, or the like.

In an embodiment, as shown in FIG. 2C, a first, or port, gasket 120 can substantially define a disc shape extending along a plane extending perpendicular to the central longitudinal axis 60. The first gasket 120 can include a lumen 124 configured to receive a portion of the stem 80 therethrough. In an embodiment, an outer diameter (d1) of the first gasket 120 can be equal to or less than an outer diameter (d2) of the cathlock 100.

In an exemplary method of use, as shown in FIGS. 2B-2F, a first gasket 120 can be disposed over the port stem 80 (FIG. 2C). The catheter 90 can be urged axially over the stem 80 of the port 70, to engage the stem 80 in an interference fit. The cathlock 100 can be slidably engaged over the catheter 90 to compress a proximal portion of the catheter 90 on to the stem 80. The cathlock 100 can be urged axially until a proximal end 108 of the cathlock 100 compresses the first gasket 120 against the port body 72, transitioning the first gasket 120 from the undeformed state (FIG. 2D) to a deformed state (FIGS. 2E-2F). In an embodiment, the cathlock 100 can compress the first gasket 120 along a first axis (e.g. the longitudinal axis) to elastically deform the first gasket 120 along a second axis extending at an angle thereto (e.g. the lateral axis, transverse axis, or an axis extending therebetween) and transition the first gasket 120 from the undeformed state to the deformed state. In an embodiment, the cathlock 100 can elastically deform the first gasket 120 from the deformed state, until an outer diameter (d1) of the first gasket 120 is equal to (FIG. 2E) or larger than (FIG. 2F) an outer diameter (d2) of the cathlock 100 in the deformed state.

In an embodiment, the first gasket 120 in the deformed state can form a continuous outer profile between the cathlock 100 and the port body 72. For example, by compressing the first gasket 120 along a first axis, and transitioning the first gasket 120 from the undeformed state to the deformed state, an outer diameter of the first gasket 120 can expand along a second axis to fill a gap (a). An outer surface of the gasket 120 can expand to align with an outer surface of the cathlock 100 providing a level surface therebetween (FIG. 2E). In an embodiment, the outer surface of the gasket 120 can expand to extend radially outwards of an outer surface of the cathlock 100 providing a convex surface therebetween (FIG. 2E). In an embodiment, the outer surface of the gasket 120 can expand to, or slightly less than, an outer surface of the cathlock 100. As such, although the first gasket 120 in the deformed state forms a slightly concave surface therebetween the surface is still considered substantially continuous since a depth of the gap (a) (i.e. perpendicular to the longitudinal axis) has been reduced to substantially less than double the width of the gap (a).

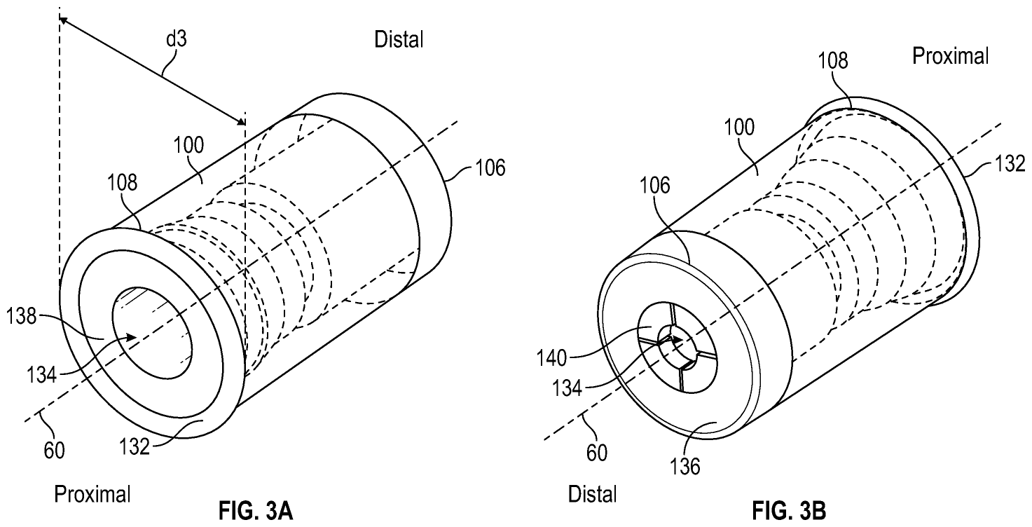

In an embodiment, as shown in FIGS. 2B, 2D-2F, and 3A-3B, a second gasket 130, or cathlock gasket 130, can be disposed on a portion of the cathlock 100, for example on an inner surface of the cathlock lumen 104. In an embodiment, the second gasket 130 can extend from a distal end 106 to a proximal end 108 of the cathlock 100. In an embodiment, the second gasket 130 can define a lumen 134 extending axially and configured to receive one or more of the catheter 90, stem 80, or portions thereof, therethrough.

In an embodiment, a diameter of the gasket lumen 134 can be equal to or less than an outer diameter of the catheter 90. In an embodiment, the lumen 134 of the second gasket 130 can elastically deform radially outward from an undeformed state to a deformed state to receive the catheter 90 therethrough and fit tightly thereon. As such, the second gasket 130 can form a continuous outer profile between the catheter 90 and the cathlock 100. For example, the second gasket 130 can form a surface extending perpendicular from the surface of the catheter 90 to a proximal end 106 of the cathlock 100, eliminating any gap (a) extending between the inner surface of the cathlock 100 and an outer surface of the catheter 90. In an embodiment, the second gasket 130 can include a valve 140 disposed at a distal end 136 thereof, and configured to provide a seal between the second gasket 130 and the outer surface of the catheter 90 to provide a continuous outer profile therebetween.

In an embodiment, a proximal end 138 of the second gasket 130 can include a flange 132 extending radially therefrom. In an embodiment, the flange 132 can define an outer diameter (d3). The outer diameter (d3) of the flange 132 can be less than, equal to, or larger than an outer diameter (d2) of the cathlock 100. In an embodiment, the flange 132 can be compressed between the cathlock 100 and one or both of the port body 72 and the first gasket 120 to transition the flange from an undeformed state to a deformed state and provide a continuous outer profile therebetween, as described herein.

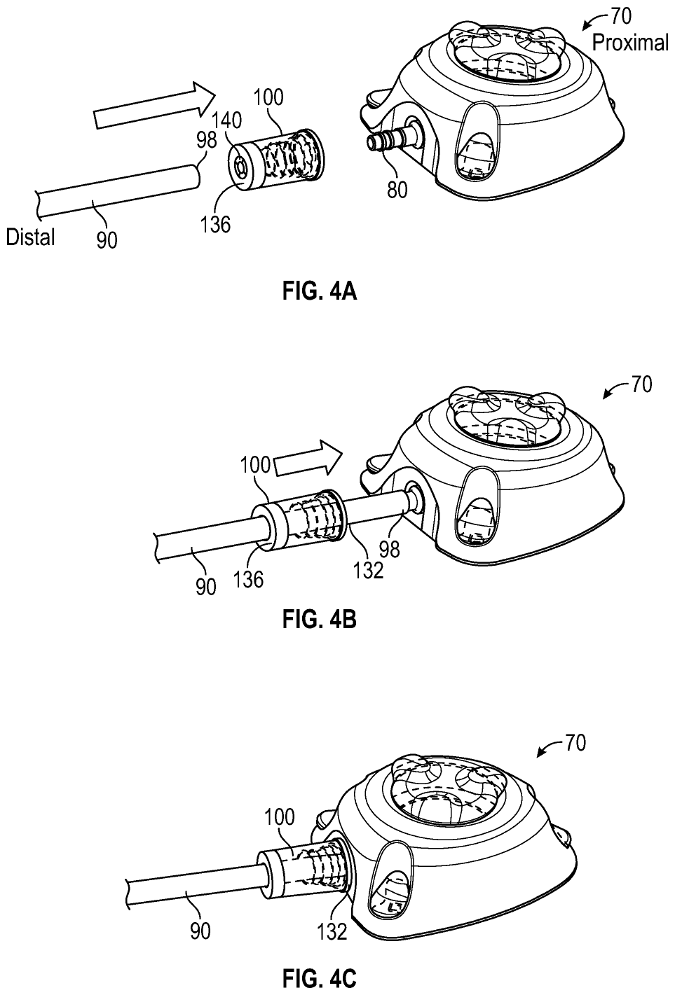

In an exemplary method of use, as shown in FIGS. 4A-4C, a catheter 90 can be urged axially to engage the stem 80 in an interference fit. The cathlock 100 can then be urged axially over a proximal portion of the catheter 90, disposed over the stem 80. In an embodiment, a distal end 136 of the gasket 130 can transition to a deformed state by stretching radially outward, forming a tight seal, and providing a continuous outer profile, between the catheter 90 and the cathlock 100. In an embodiment, a valve 140 disposed at a distal end 136 of the gasket 130 can form a continuous outer profile between the catheter 90 and the cathlock 100.

Urging the cathlock 100 axially can compress one or both of a proximal end 138 of the second gasket 130 and the flange 132 against the port body 72. The cathlock 100 can elastically deform one or both of a proximal end 138 of the second gasket 130 and the flange 132 from an undeformed state to a deformed state to form a continuous outer profile between the cathlock 100 and the port body 72, as described herein. In an embodiment, the second gasket 130 can engage a first gasket 120 coupled to the port 70. The second gasket 130 and the first gasket 120 can co-operate to form a continuous outer profile between the cathlock 100 and the port body 72.

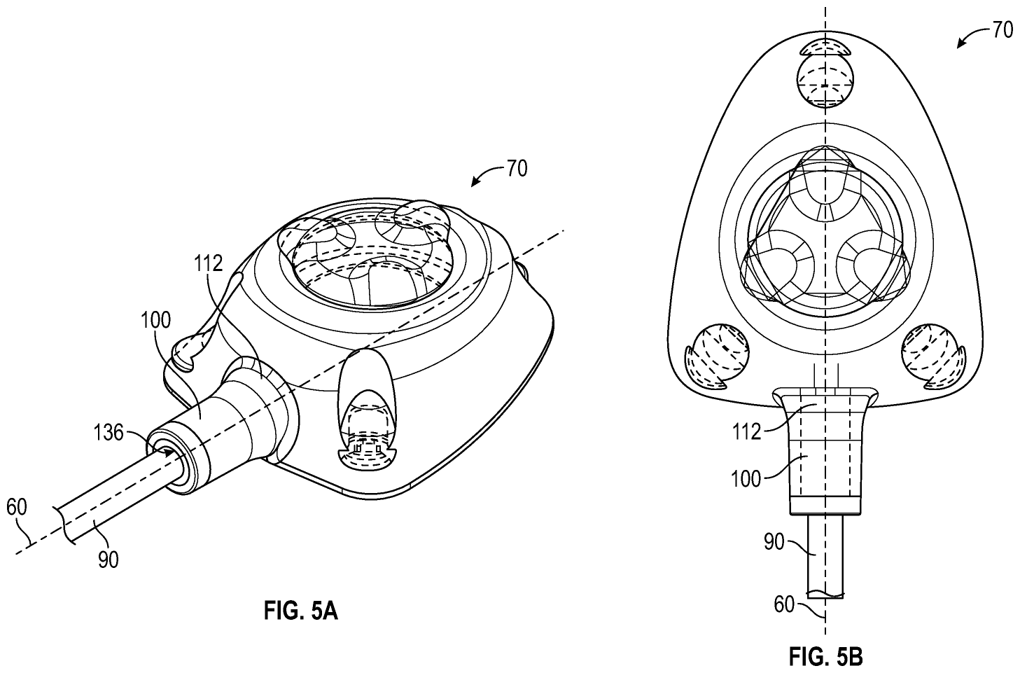

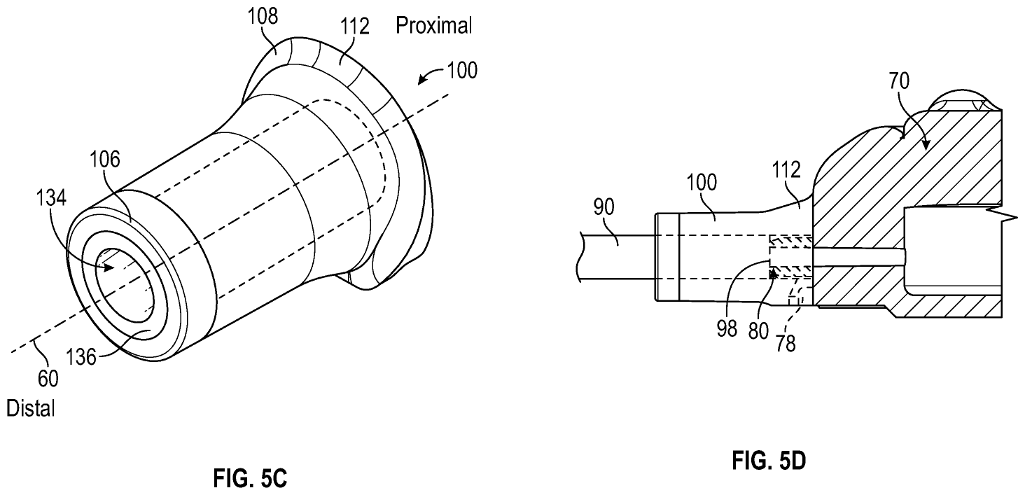

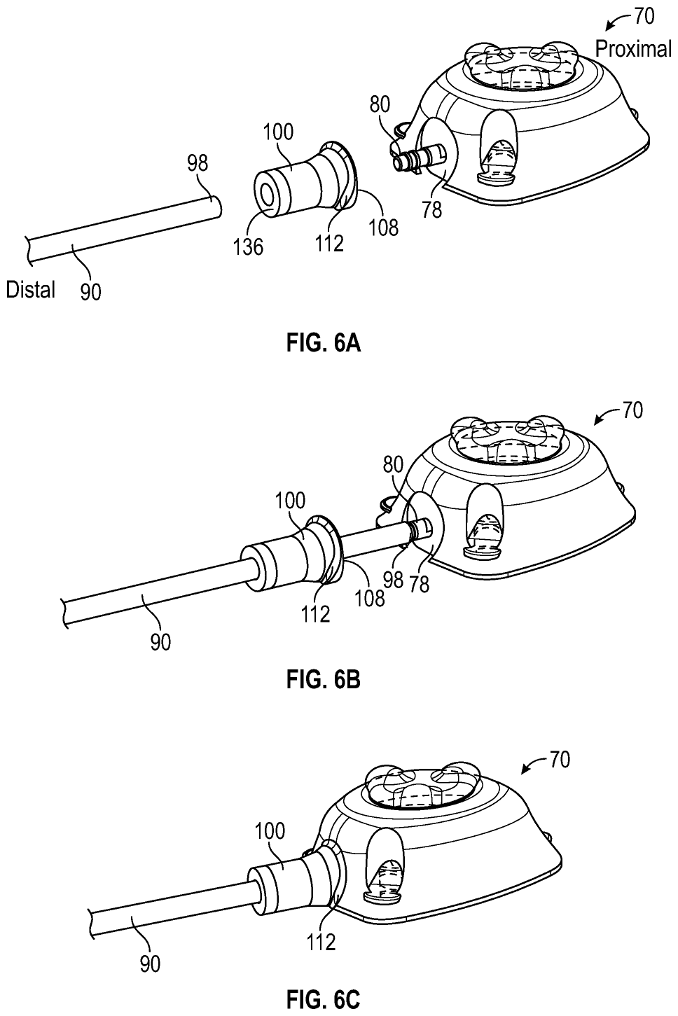

In an embodiment, as shown in FIGS. 5A-6C, a proximal end 108 of the cathlock 100 can include a flared portion 112. The flared portion 112 can be configured to engage a recess 78 disposed in the port body 72. The flared portion 112 can extend annularly about a portion of the cathlock 100, about the central longitudinal axis 60. The recess 78 can extend annularly about a portion of the stem 80, about the central longitudinal axis 60. The outer surface of the flared portion 112 can co-ordinate with an outer surface of the port body 72 to define a continuous outer profile (FIG. 5A).

In an exemplary method of use, as shown in FIGS. 6A-6C, a catheter 90 can be urged axially to engage the stem 80 in an interference fit. The cathlock 100 can then be urged axially over a proximal portion of the catheter 90, disposed over the stem 80. In an embodiment, a distal end 136 of the gasket 130 can form a continuous outer profile between the catheter 90 and the cathlock 100. In an embodiment, a valve 140 disposed at a distal end 136 of the gasket 130 can form a continuous outer profile between the catheter 90 and the cathlock 100.

Urging the cathlock 100 axially can engage a proximal end 108 of the cathlock 100 within the recess 78 of the port 70. In an embodiment, the port 70 can further include a first gasket 120 disposed about the stem 80 and disposed between the cathlock 100 and the port body 72, as described herein. As shown in FIG. 6C, an outer profile of the flared portion 112 can co-ordinate with and outer profile of the port body 72 to define a continuous outer surface therebetween and can mitigate tissue ingrowth between the port body 72 and the cathlock 100.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A cathlock system, comprising:
a port including a stem extending therefrom;
a catheter defining a lumen and configured to engage the stem;
a cathlock configured to encircle a portion of one or both of the stem and the catheter, the cathlock having a distal end including a cathlock gasket integral with the cathlock or coupled to the cathlock; and
a disk-shaped port gasket, each of the port gasket and the cathlock gasket formed of a compliant material and configured to elastically transition between an undeformed state and a deformed state having a continuous outer profile between an outer surface of the cathlock and one or both of the catheter and the port.

2. The cathlock system according to claim 1, wherein the port gasket is coupled with the port body and extends annularly about the stem.

3. The cathlock system according to claim 1, wherein the port gasket extends along a plane perpendicular to a longitudinal axis of the stem, and an outer diameter of the gasket in the undeformed state is equal to, or less than, an outer diameter of the cathlock.

4. The cathlock system according to claim 3, wherein the outer diameter of the port gasket in the deformed state is equal to or larger than the outer diameter of the cathlock.

5. The cathlock system according to claim 1, wherein the outer diameter of the port gasket in the deformed state is larger than the outer diameter of the port gasket in the undeformed state.

6. (canceled)

7. The cathlock system according to claim 1, wherein the cathlock gasket further includes a flange extending radially outward from a proximal end of the cathlock gasket.

8. The cathlock system according to claim 7, wherein the flange defines a first diameter in the undeformed state, and a second diameter in the deformed state, the second diameter being larger than the first diameter.

9. The cathlock system according to claim 8, wherein the first diameter of the flange is less than an outer diameter of the cathlock, and the second diameter of the flange is equal to, or larger than the outer diameter of the cathlock.

10. The cathlock system according to claim 1, wherein the cathlock is formed of a first material and each of the cathlock gasket and the port gasket is formed of the compliant material as a second material different from the first material.

11. The cathlock system according to claim 10, wherein the first material defines a first durometer and the second material defines a second durometer, the second durometer being less than the first durometer.

12. The cathlock system according to claim 10, wherein the first material is a substantially rigid material and the second material is an elastically deformable material.

13. The cathlock system according to claim 10, wherein the first material includes one of a plastic, polymer, metal, alloy, or composite, and the second material includes one of a plastic, polymer, elastomer, rubber, thermoplastic elastomer, or silicone rubber.

14. The cathlock system according to claim 1, wherein each of the cathlock gasket and the port gasket is formed of a biocompatible material, and wherein the cathlock is configured to mitigate tissue ingrowth between the cathlock and one or both of the catheter and the port.

15. The cathlock system according to claim 1, the port gasket further including a valve disposed between an outer surface of the catheter and an inner surface of the cathlock configured to provide a fluid tight seal therebetween.

16. A system for coupling a catheter to a subcutaneous vascular access device, comprising:
a body of the subcutaneous vascular access device having a recess disposed therein;
a stem extending longitudinally from the recess;
a catheter defining a lumen and configured to engage the stem;
a cathlock configured to encircle a portion of one or both of the stem and the catheter, a proximal end of the cathlock configured to fit within the recess, and a distal end of the cathlock including a cathlock gasket integral with the cathlock or coupled to the cathlock; and
a disk-shaped port gasket, each of the port gasket and the cathlock gasket formed of a compliant material and configured to elastically transition between a deformed state and an undeformed state having a continuous outer profile between an outer surface of the cathlock and the catheter.

17. The system according to claim 16, wherein the cathlock includes a flared portion disposed at a proximal end of the cathlock.

18. The system according to claim 17, wherein the flared portion co-operates with an outer surface of the body to define a continuous outer profile when the proximal end of the cathlock is disposed within the recess.

19. (canceled)

20. The system according to claim 16, wherein the cathlock gasket is integral with the cathlock.

21. The system according to claim 16, wherein the cathlock gasket is formed as a separate structure and coupled with the cathlock with an interlocking fit, interference fit, press-fit, or snap fit engagement, adhesive, bonding, or welding.

22. The system according to claim 16, wherein the cathlock is formed of a first material and each of the cathlock gasket and the port gasket is formed of the compliant material as a second material different from the first material.

23. The system according to claim 22, wherein the first material defines a first durometer and the second material defines a second durometer, the second durometer being less than the first durometer.

24. The system according to claim 16, the port gasket further including a valve disposed between an outer surface of the catheter and an inner surface of the cathlock configured to provide a fluid tight seal therebetween.

25-33. (canceled)
